(19) United States
(12) Patent Application Publication
(10) Pub. No.: US 2025/0135412 A1
(43) Pub. Date: May 1, 2025

(54) CONTRAST AGENT MIXER

(71) Applicant: Lument AB, Lund (SE)

(72) Inventors: Thomas FORK, Malmö (SE); Ingvar ADNERHILL, Malmö (SE); Lucia CASAL-DUJAT, Dalby (SE); Wenyun LIU, Åkarp (SE); Olof BÖÖK, Lund (SE)

(73) Assignee: Lument AB, Lund (SE)

(21) Appl. No.: 18/694,203

(22) PCT Filed: Sep. 22, 2022
(86) PCT No.: PCT/SE2022/050833

§ 371 Date: Mar. 21, 2024

(30) Foreign Application Priority Data
Sep. 23, 2021 (SE) 2151163-9
Sep. 30, 2021 (SE) 2151200-9
Sep. 15, 2022 (SE) 2251068-9
Sep. 15, 2022 (SE) 2251071-3

(51) Int. Cl. B01F 23/53; B01F 23/50; B01F 27/806; B01F 27/93; B01F 35/221; B01F 35/222; B01F 35/32; B01F 35/33; B01F 101/00

(52) U.S. Cl. CPC B01F 23/53; B01F 23/582; B01F 27/806; B01F 27/93; B01F 35/221422; B01F 35/222; B01F 35/323; B01F 35/332; B01F 2101/2202

(57) ABSTRACT

A contrast agent mixer (100) for providing a foam type contrast agent is presented. The mixer (100) comprises a holding arrangement (109) for supporting a mixing container, a substantially homogeneous circular mixer blade (110) for mixing a contrast powder with a liquid in the mixing container (200), and a controller configured to control a rotational speed of the mixer blade (110) and a vertical distance (D) between the holding arrangement (109) and the mixer blade (110). A method, a system and a mixing container are also presented.

### TECHNICAL FIELD

The present invention relates to mixer and more precisely to a contrast agent mixer suitable for providing a per-oral negative contrast agent foam and associated method and system.

### BACKGROUND

Computerized tomography (CT) is a diagnostic imaging technique that creates detailed images of a body, e.g., a human body, with its interior by combining series of X-ray captions that create cross-sectional images or slices of parenchymal organs, muscles, fat tissue, bones, vessels, lymph nodules, etc. in health and disease. Today CT is a frequently used tool due to its lower cost and notably faster examinations compared to other tomography techniques such as magnetic resonance tomography, and also due to its higher availability worldwide. In addition to its common use in diagnosing cancer, CT is widely used to facilitate diagnosing a variety of other diseases and disorders, such as inflammatory diseases, trauma, anomalies, etc.

In CT of the abdomen (CT-abd), contrast agents are used for demarcating structures by increasing differences in density between tissue compartments. The enhanced difference in contrast improves visualization of details necessary for the radiologist to detect and follow abnormalities within the abdomen and pelvis over time and thus, with a possible medical diagnosis. The radiodensity of structures and/or materials is measured in Hounsfield Units (HU). The abdominal organs and structures are displayed in a variety of colors in the grey scale depending on the radiodensity of their composition, from white (such as bones; around +1000 HU), to light grey (such as blood vessels; around +70 HU), and black (such as air in the lungs; around −1000 HU).

A patient routinely referred to CT-abd is usually prepared with a per-oral agent for demarcating the gastro-intestinal tract. Up until now, the most commonly used demarcating agent has been a diluted solution of an iodine contrast medium meant for intra-venous application, resulting in a white bowel content, i.e., with positive HU. Other agents are iso-osmotic solutions that provide densities of around 10 HU, exhibiting bowel lumen in grey, close to the color of other body structures. Thus, positive oral filling agents provide no or unsatisfactory contrast between the bowel wall and the lumen of the small intestine on CT images. As a consequence, images of the bowel wall are less easy to read which may result in radiological diagnoses of reduced quality, including both false positive and negative diagnoses. Consequently, a negative, “black” filling, contrast agent, with notably larger contrast against the mucosal lining and of the gut wall, was introduced in EP 3589331 thereby creating an opportunity for improved medical evaluation.

The contrast agent introduced in EP 3589331 is a fluid, aqueous foam of microbubbles. The foam is created from a dispersion obtained by stirring, manually or by magnetic stirrer, a contrast powder with a liquid until a completely homogenous dispersion was obtained. This dispersion was mixed into a foam by manually using a blender. A blade of the blender is continuously kept in the dispersion without creating any air pocket to avoid the incorporation of extra air and the formation of new big bubbles. The foam is whipped until the foam is homogenous and with no visible bubbles. In case visible bubbles are detected by bear eye at the surface of the foam, the bubbles are removed with a spoon or with a suction device such as a Pasteur pipette. If too many bubbles that may not be removed are present at the surface and/or in the bulk, the foam will have to be discarded or re-whipped increasing the preparation time and the cost of the product.

The process of producing a contrast agent foam is sensitive. Excess air will cause an increase in overrun and consequently produce a thicker foam, as well as the formation of big bubbles giving a non-homogenous and high polydispersity foam. Such a foam would negatively interfere with the quality of the x-ray images. The foam should not comprise any clearly visible bubbles among the microbubbles that the foam is made up of.

Thus, from the above it is understood that there is room for improvements.

### SUMMARY

An object of the present invention is to provide a new type of mixer which is improved over prior art and which addresses or at least mitigates the drawbacks discussed above. More specifically, an object of the invention is to provide a contrast agent mixer suitable for providing a per-oral negative contrast agent foam for e.g., abdominal computer tomography. These objects are addressed by the technique set forth in the appended independent claims with preferred embodiments defined in the dependent claims related thereto.

In a first aspect, a contrast agent mixer for providing a foam type contrast agent is presented. The mixer comprises a holding arrangement for supporting a mixing container, a substantially homogeneous circular mixer blade for mixing a contrast powder with a liquid in the mixing container, and a controller configured to control a rotational speed of the mixer blade and a vertical distance between the holding arrangement and the mixer blade.

In one variant, the holding arrangement is movable by a second motor arrangement of the mixer to control the vertical distance between the holding arrangement and the mixer blade. The second motor arrangement is controlled by the controller. This is beneficial as it reduces noise and vibrations during mixing and controlling of the distance between the mixer blade and the holding arrangement.

In one variant, the mixer blade is arranged on a mixer shaft rotatable about a longitudinal axis of the mixer shaft by a first motor arrangement controlled by the controller. This is beneficial as motor arrangements provide a controllable torque, are energy efficient, silent, cost effective and comparably easy to control.

In one variant, the mixer blade is movable by the second motor arrangement of the mixer to control the vertical distance between the holding arrangement and the mixer blade. The second motor arrangement is controlled by the controller. This is beneficial as the cost of the mixer may be reduced to less stringent requirement on the second motor arrangement.

In one variant, the mixer blade is operatively connected to the first motor arrangement by means of a clutch. Having a clutch is beneficial as it allows for the removal of the mixer blade from the mixer to simplify replacement and/or cleaning of the mixer blade.

In one variant, the clutch is a magnetic clutch comprising an upper member operatively connected to the first motor arrangement and a lower member operatively connected to the mixer blade. The upper member is connected to the lower member by means of one or more magnets. Using magnets for the clutch is beneficial as it allows the tool-less removal of the mixer blade, it provides an improved user experience and it reduces the time and effort needed when replacing and/or cleaning the mixer blade.

In one variant, the lower member or the upper member of the clutch is concavely formed, and the other of the lower member or the upper member of the clutch is matingly convexly formed. This is beneficial as it allows for easy and correct positioning of the mixer blade to the first motor arrangement reducing the time and effort needed when replacing and/or cleaning the mixer blade.

In one variant, the mixer blade is arranged on the mixer shaft such that a blade angle is formed between a plane of the mixer blade and a reference plane perpendicular to a longitudinal axis of the mixer shaft. The blade angle is in the range of 0.5 to 5°, preferably in the range of 2 to 4°. This is beneficial as the blade angle can be used to control the amount of air incorporated in the foam and thereby the volume of the foam. The blade angle increases the effectiveness of the mixer blade.

In one variant, the negative oral contrast agent mixer further comprises a liquid container arranged to dispense the liquid into the mixing container. This is beneficial as a used does not have to ensure that liquid is in the mixing container before starting the mixer.

In one variant, the negative oral contrast agent mixer further comprises a valve arranged in a fluid pathway between the liquid container and the mixing container and controllable between an open position and a closed position by the controller. This is beneficial as the controller may control when the liquid is added to the mixing container and/or the amount of liquid added to the mixing container.

In one variant, the negative oral contrast agent mixer further comprises a powder container arranged to dispense the contrast powder into the mixing container. This is beneficial as a used does not have to ensure that contrast powder is in the mixing container before starting the mixer.

In one variant, the negative oral contrast agent mixer further comprises a powder dispenser controllable, between an open position and a closed position by the controller. This is beneficial as the controller may control when the contrast powder is added to the mixing container and/or the amount of contrast powder added to the mixing container.

In a second aspect, a method for providing a per-oral negative contrast agent foam for abdominal CT is presented. The method is performed by the controller of the negative oral contrast agent mixer according to the first aspect. The mixer blade is configurable to mix a contrast powder with a liquid in a mixing container. The method comprises controlling the mixer blade to rotate at a wanted rotational speed, and controlling a vertical distance between the mixer blade and the holding arrangement repeatedly change between an upper distance and a lower distance.

In one variant, wherein the contrast agent mixer comprises a liquid container arranged to dispense liquid into the mixing container by means of a valve, the method further comprises controlling the valve to dispense liquid into the mixing container. This is beneficial as the controller may control when the liquid is added to the mixing container and/or the amount of liquid added to the mixing container.

In one variant, controlling the mixer blade to rotate at the wanted rotational speed is initiated before controlling the valve to dispense liquid into the mixing container. This is beneficial as the addition of water during rotation of the mixer blade reduces the risk of the contrast powder forming lumps in the liquid.

In one variant, controlling the mixer blade to rotate at the wanted rotational speed is initiated after controlling the valve to dispense the liquid into the mixing container. This is beneficial as it reduces a risk of spill and and/or splashes.

In one variant, wherein the contrast agent mixer comprises a powder dispenser arranged to dispense contrast powder into the mixing container by means of a powder dispenser, the method further comprises controlling the powder dispenser to dispense the powder into the mixing container. This is beneficial as the controller may control when the contrast powder is added to the mixing container and/or the amount of contrast powder added to the mixing container.

In one variant, controlling the vertical position of the distance between the mixer blade and the holding arrangement to repeatedly change between the upper distance and the lower distance is performed at least 8 times, preferably at least 12 times. This is beneficial as it provides a foam especially suitable for per-oral negative contrast agent foam for abdominal CT.

In one variant, controlling the mixer blade to rotate at the wanted rotational speed further comprises, during controlling the vertical distance between the mixer blade and the holding arrangement, stopping the rotation of the mixer blade for a rest period. This is beneficial as it allows for comparably large bubbles to rise to a surface of the mixing container where they will collapse, either by contact with the surface of when the rotation of the mixer blade is commenced.

In a third aspect, a mixer system comprising the negative oral contrast agent mixer of the first aspect, a mixing container, a contrast powder and a liquid. The mixing container is arrangeable to receive the mixer blade of the mixer.

In one variant, a diameter of the mixer blade is less than half a diameter of the mixing container and larger than one third of the diameter of the mixing container. This is beneficial as it provides a foam especially suitable for per-oral negative contrast agent foam for abdominal CT.

In one variant, the vertical distance between the mixer blade and the holding arrangement is controllable between an upper distance and a lower distance. The mixer blade is adjacent to a bottom of the mixing container at the lower position. This is beneficial as it allows for the production of a homogenous foam without enlarged bubbles or residue of the contrast powder.

In one variant, the upper distance between the mixer blade and the holding arrangement is, during operation of the mixer, below 75% of a wanted height of the negative contrast agent foam in the mixer container, preferably below 65% of the wanted height of the negative contrast agent foam in the mixer container, and most preferably below 55% of the wanted height of the negative contrast agent foam in the mixer container. This is beneficial as it allows for the production of a homogenous foam without enlarged bubbles or residue of the contrast powder.

In one variant, the contrast powder is an egg albumen powder.

In one variant, the liquid is water.

In a fourth aspect, a negative oral contrast agent mixer for providing a per-oral negative contrast agent foam for abdominal computer tomography, CT, is presented. The mixer comprises a mixer blade for mixing a contrast powder with a liquid in a mixing container, and a controller configured to control a vertical position of the mixer blade and a rotational speed of the mixer blade.

In one variant, the mixer blade is arranged on a mixer shaft rotatable about a longitudinal axis of the mixer shaft by a first electrical motor and movable along the longitudinal axis of the mixer shaft by a second electrical motor. The first electrical motor and the second electrical motor are controlled by the controller. This is beneficial as electrical motors provide a controllable torque, are energy efficient, silent, cost effective and comparably easy to control.

In one variant, the mixer blade is operatively connected to the first electrical motor by means of a clutch. Having a clutch is beneficial as it allows for the removal of the mixer blade from the mixer to simplify replacement and/or cleaning of the mixer blade.

In one variant, the clutch is a magnetic clutch comprising an upper member operatively connected to the first electrical motor and a lower member operatively connected to the mixer blade. The upper member is connected to the lower member by means of one or more magnets. Using magnets for the clutch is beneficial as it allows the tool-less removal of the mixer blade, it provides an improved user experience and it reduces the time and effort needed when replacing and/or cleaning the mixer blade.

In one variant, the lower member or the upper member of the clutch is concavely formed, and the other of the lower member or the upper member of the clutch is matingly convexly formed. This is beneficial as it allows for easy and correct positioning of the mixer blade to the first electrical motor reducing the time and effort needed when replacing and/or cleaning the mixer blade.

In one variant, the mixer blade is arranged on the mixer shaft such that a blade angle is formed between a plane of the mixer blade and a reference plane perpendicular to a longitudinal axis of the mixer shaft. The blade angle is in the range of 0.5 to 5°, preferably in the range of 2 to 4°. This is beneficial as the blade angle can be used to control the amount of air incorporated in the foam and thereby the volume of the foam. The blade angle increases the effectiveness of the mixer blade.

In one variant, the mixer blade is a homogenous substantially circular mixer blade. This is beneficial as it reduces the risk of turbulence in the mixing process and provides a foam with a more homogenous bubble size.

In one variant, the negative oral contrast agent mixer further comprises a liquid container arranged to dispense the liquid into the mixing container. This is beneficial as a used does not have to ensure that liquid is in the mixing container before starting the mixer.

In one variant, the negative oral contrast agent mixer further comprises a valve arranged in a fluid pathway between the liquid container and the mixing container and controllable between an open position and a closed position by the controller. This is beneficial as the controller may control when the liquid is added to the mixing container and/or the amount of liquid added to the mixing container.

In one variant, the negative oral contrast agent mixer further comprises a powder container arranged to dispense the contrast powder into the mixing container. This is beneficial as a used does not have to ensure that contrast powder is in the mixing container before starting the mixer.

In one variant, the negative oral contrast agent mixer further comprises a powder dispenser controllable, by the controller, into at least dispensing position at which contrast powder is dispensed into the mixing container. This is beneficial as the controller may control when the contrast powder is added to the mixing container and/or the amount of contrast powder added to the mixing container.

### BRIEF DESCRIPTION OF THE DRAWINGS

Embodiments of the invention will be described in the following; references being made to the appended diagrammatical drawings which illustrate non-limiting examples of how the inventive concept can be reduced into practice.

FIG. 1a is a perspective view of a contrast agent mixer according to some embodiments of the present invention;

FIG. 1b is side view of a contrast agent mixer according to some embodiments of the present invention;

FIG. 1c is a perspective view of a contrast agent mixer according to some embodiments of the present invention;

FIGS. 1d-e are side views of contrast agent mixers according to some embodiments of the present invention;

FIGS. 2a-d are perspective views of a clutch according to some embodiments of the present invention;

FIG. 3a is a side view of a mixer blade according to some embodiments of the present invention;

FIG. 3b is a perspective view of a mixer blade according to some embodiments of the present invention;

FIG. 4a is a front view of a mixing container with a mixer blade according to some embodiments of the present invention;

FIG. 4b is a time series plot of vertical movement of a mixer blade according to some embodiments of the present invention;

FIG. 5 is a partial side view of a mixer blade according to some embodiments of the present invention;

FIG. 6 is a top view of a mixer blade and a mixing container according to some embodiments of the present invention;

FIG. 7 is a perspective view of a contrast agent mixer according to some embodiments of the present invention;

FIGS. 8a-b are perspective views of a contrast agent mixer according to some embodiments of the present invention;

FIG. 8c is a partial block diagram of a contrast agent mixer according to some embodiments of the present invention

FIG. 9 is a schematic block view of a mixer system according to some embodiments of the present invention; and

FIG. 10 is a schematic block view of a method according to some embodiments of the present invention.

### DETAILED DESCRIPTION OF EMBODIMENTS

Hereinafter, certain embodiments will be described more fully with reference to the accompanying drawings. The invention may, however, be embodied in many different forms and should not be construed as limited to the embodiments set forth herein; rather, these embodiments are provided by way of example so that this disclosure will be thorough and complete, and will fully convey the scope of the invention, such as it is defined in the appended claims, to those skilled in the art.

The term “coupled” is defined as connected, although not necessarily directly, and not necessarily mechanically. Two or more items that are “coupled” may be integral with each other. The terms “a” and “an” are defined as one or more unless this disclosure explicitly requires otherwise. The terms “substantially”, “approximately”, and “about” are defined as largely, but not necessarily wholly what is specified, as understood by a person of ordinary skill in the art. The terms “comprise” (and any form thereof, such as “comprises” and “comprising”), “have” (and any form thereof, such as “has” and “having”), “include” (and any form thereof, such as “includes” and “including”) and “contain” (and any form thereof, such as “contains” and “containing”) are open-ended linking verbs. As a result, a method that “comprises”, “has”, “includes” or “contains” one or more steps possesses those one or more steps, but is not limited to possessing only those one or more steps.

Systems composed of air dispersion in aqueous media provide negative density contrast values in the range of −1000 HU to 0 HU, depending on the proportion of dispersed air, and may thus be used as negative contrast agents. Such a contrast agent is usable for MRI, ultrasound and CT. As it is a food based contrast agent, it is particularly suitable for per-oral administration and therefor for abdominal imaging. The present disclosure is applicable for providing foam type contrast agents for all of these applications, positive and negative, regardless of use. The disclosure is focused on a per-oral contrast agent foam for abdominal CT imaging CT but this is but one exemplary embodiment. The teachings of the present disclosure are also applicable when providing other filling agents with or without (negative) contrast agent properties on radiological images from abdominal CT-scans. For abdominal CT imaging, the negative density contrast values provided should preferably be in the range −300 to −800 HU corresponding to a fairly high volume proportion of air. Further, a contrast agent for use in CT imaging should be sufficiently stable in the gastrointestinal tract to provide essentially the same CT negative density contrast values throughout the gastrointestinal tract. As recognized in the art, dispersion of air in liquids, i.e., foams, may be provided by whipping or beating an aqueous solution or dispersion, comprising a foaming agent. Typical examples of foaming agents are detergents. The type and amount of the foaming agents will affect properties of the final foam. Further, also the amount of air incorporated into the aqueous solution or dispersion will influence in the properties of the final foam. The preparation of a negative contrast agent for abdominal CT involves stirring a dry powder 5 (see FIG. 9), further contrast powder 5 or powder 5, with a liquid to obtain a dispersion, typically using a magnetic stirrer. This dispersion is mixed, generally with a blender, to incorporate air into the dispersion thereby creating a foam that is orally administered to a patient. In order to arrive at repeatable and comparable results from e.g., CT involving the negative contrast agent, the foam must be consistent regardless of who, where or when the foam is prepared. In addition to this, the use of dual machinery, i.e., the magnetic stirrer and the blender, is tedious, adds time and requires several manual steps in addition to requiring two separate machines to be washed, maintained and services.

Regarding the contrast powder 5, among the food-based proteins, egg white protein has been found to have exceptional functional properties on gelation and foam formation. Egg white protein, or egg albumen, is comprised by several globular proteins (ovalbumin, ovotransferin, ovomucoid, ovomucin, lysozyme, globulin, avidin). Even though ovalbumin is one of the critical proteins, the combination of different proteins contained in egg albumen is advantageous in foaming and foam stability properties. A mixture of opposed charges and the formation of intermolecular bonds improve the stabilization of food foams. The mixture may thus preferably comprise at least ovalbumin, ovomucin and ovoglobulin. In the dispersion, the surfactant, e.g., egg albumen, permits the formation of air bubbles and stabilization thereof, due to their amphiphilic nature. Albumen proteins turned out to have exceptional functional properties on foam formation and gelation and here hence preferred. However, in order to enhance the stabilization of the dispersed air bubbles, a foam stabilizer, e.g., a hydrocolloid acting as foam stabilizer, such as natural gum should be present in the liquid composition.

Although the term “contrast powder” is used throughout this application, the powder does not need to have contrast enhancing properties itself. The term “contrast powder” refers to powder used for providing a contrast agent, or in other words, a contrast agent in powder form. As further elaborated below, the contrast powder may have stabilizing properties for a foam, wherein the air bubbles of the foam are contrast enhancing, and the contrast powder may hence be described as contrast facilitating.

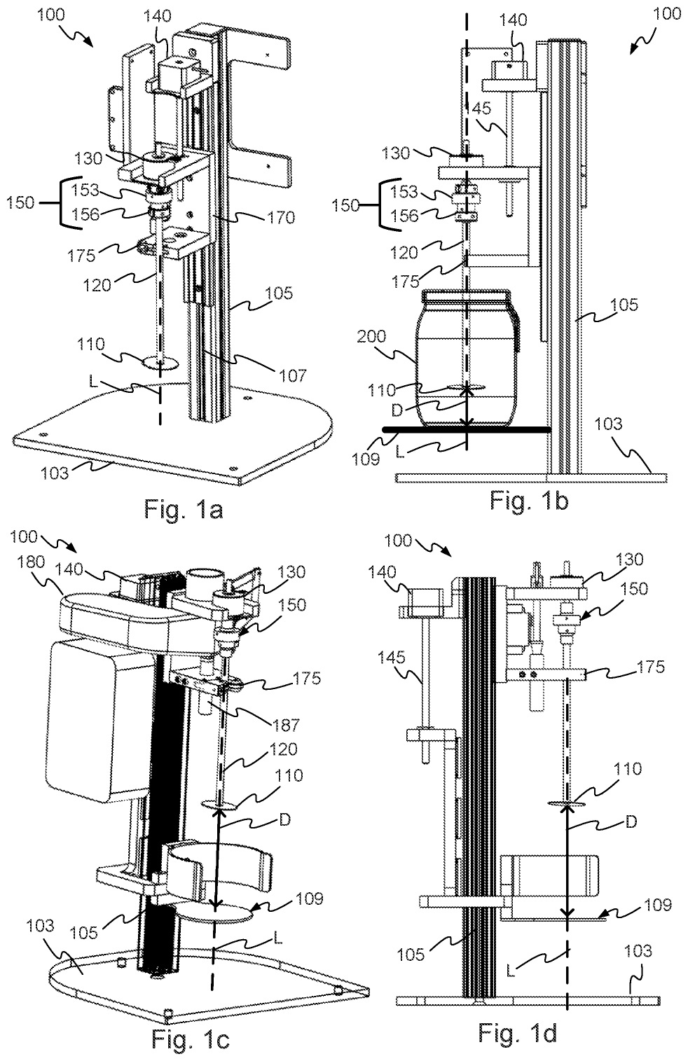

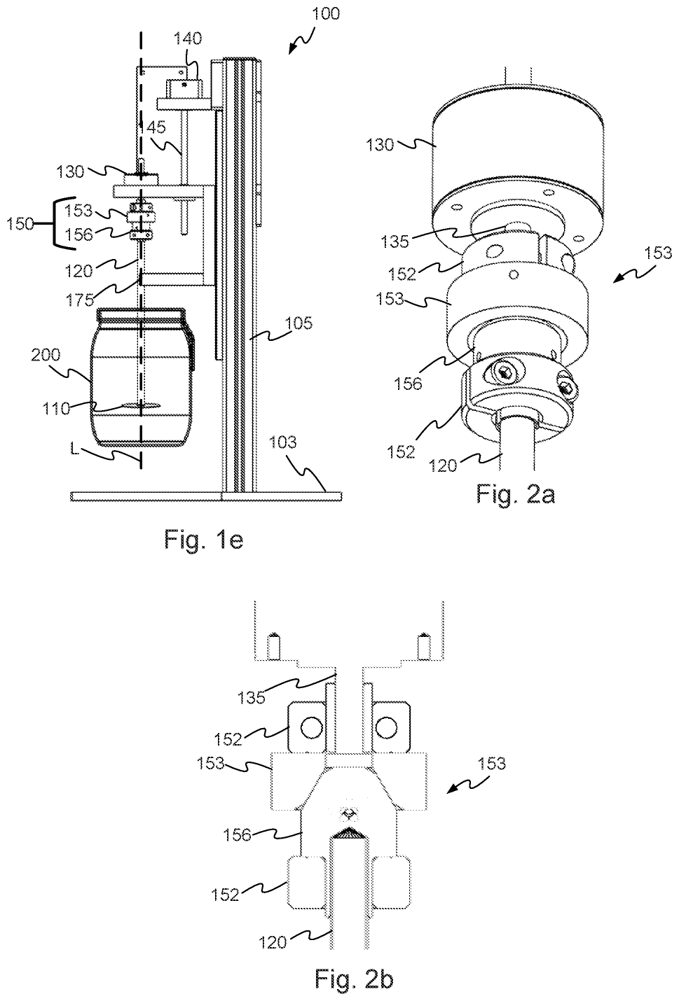

A repeatable preparation process of a negative oral contrast agent foam 7 (see FIG. 9) is provided by the negative oral contrast agent mixer 100 of FIGS. 1a and 1b. The negative oral contrast agent mixer 100, or mixer 100 for short, comprises a mixer blade 110 for providing a per-oral negative contrast agent foam 7 for abdominal computer tomography by mixing the contrast powder 5 with a liquid 3 (see FIG. 9). That is to say, the mixer 100 is configured to provide the negative contrast agent foam 7 starting from the contrast powder 5 and the liquid 3, i.e., there is no need to provide a dispersion first. The mixer 100 further comprises a controller 160 (see FIG. 9) configured to control a vertical distance L_{P} (see FIG. 4b) between the mixer blade 110 and a holding arrangement 109 (see FIG. 1b) and a rotational speed of the mixer blade 110. In some embodiments, the controller 160 is further configured to control a rotational direction of the mixer blade 110. The control of the vertical distance L_{P} between the mixer blade 110 and the holding arrangement 109 reduces a risk that comparably large air bubbles are part of the foam 7 and ensures that a homogenous foam 7 is provided. The control of the rotational speed of the mixer blade 110 enables control of the amount of air incorporated in the foam 7. An increase in rotation speed would cause more air incorporation, and thus larger foam volume, and a decrease in rotational speed would consequently incorporate less air with a reduced foam volume as a result. In this configuration, the mixer 100 enables the forming of the negative contrast agent foam 7 without having to first stir the dry powder 5 with the liquid 3 into a dispersion. In addition to greatly improving the quality of the foam 7, this significantly reduces the preparation time of the negative contrast agent foam 7 thereby saving precious time of caretakers, hospital personnel etc.

Although the present disclosure is mainly made with reference to a contrast agent mixer 100, it should be emphasized already now that such mixer may be used also for other purposes such as, but not limited to, ultrasonic gels etc. An ultrasonic gel may be composed of a mixture of propylene glycol and water and such a mixture may very well be obtained by the mixer 100 of the present disclosure.

As seen in FIG. 1b, the mixing is preferably performed in a mixing container 200 which holds the dry powder 5 and the liquid 3 that are to be mixed into the negative contrast agent foam 7. The dry powder 5 and/or the liquid 3 may be manually added to the mixing container 200 by a user of the mixer 100 prior to starting the mixer 100, or, as will be explained in further detail in other sections of this disclosure, be automatically or semi-automatically added to the mixing container 200 by the mixer 100. The mixing container may be supported by holding arrangement 109 that may, as will be further explained, be fixed or movable along the longitudinal axis of the mixer shaft L.

The mixer blade 110 is preferably arranged at one end of a mixer shaft 120 of the mixer 100. The mixer shaft 120 may be formed as an integral part of the mixer blade 110, or the mixer blade 110 may be attached to the mixer shaft by welding or a suitable attachment means such as one or more screws, pins etc. In some embodiments, the mixer blade 110 is removable from the mixer shaft 120. The mixer shaft 120 is rotatable about a longitudinal axis L of the mixer shaft 120 by means of a first motor arrangement 130. That is to say, the mixer blade 110 is operatively connected to the first motor 130. A rotational speed of the first motor 130 preferably determines the rotational speed of the mixer blade 110 and the rotational speed of the mixer blade 110 is preferably controlled by the controller 160 controlling the rotational speed of the first motor 130. The operative connection between the first motor 130 and the mixer blade 110 may comprise one or more transmissions (not shown) in order to assist in controlling the rotational speed and/or a torque of the mixer blade 110.

As previously mentioned, the vertical distance L_{P} between the mixer blade 110 and the holding arrangement 109 is controlled by the controller 160. Preferably, the mixer 100 further comprises a second motor arrangement 140 arranged to move the mixer blade 110, or as will be explained with reference to FIG. 1c, the holding arrangement 109 along the longitudinal axis L of the mixer shaft 120. In this embodiment, the second motor arrangement 140 may be arranged to move the mixer blade 110 along the longitudinal axis L of the mixer shaft 120 in any suitable way such that a distance D between the holding arrangement 109 and the mixer blade 110 is changed. In one further embodiment, the mixer blade 110 is moved together with the first motor arrangement 130. This may be provided by having the second motor arrangement 140 control a position of a carrier 170 of the mixer 100. The carrier 170 may in turn may be arranged to support the first motor 130 and the mixer blade 110 such that when the carrier 170 is moved, the first motor arrangement 130 and the mixer blade 110 are moved with it. The mixer 100 may further comprise a base 103 and a column 105 attached to the base 103, preferably at a substantially perpendicular angle to the base 103. The second motor arrangement 140 may be attached to the column 103 and provided with a motor shaft 145 in the form of a lead screw 145, e.g., an acme shaft, connected to the carrier 170. As the second motor arrangement 140 is actuated, the lead screw 145 is rotated and the carrier 170 is moved along the longitudinal axis L of the mixer shaft 120. In some embodiments, the column 105 may be provided with guide rails 107 and the carrier 170 may be provided with mating guides (not shown) allowing it to be guided along the column 105. The vertical distance L_{P} between the mixer blade 110 and the holding arrangement 109 along the longitudinal axis L of the mixer shaft 120 is preferably controlled by the controller 160 controlling the second motor arrangement 140.

In FIG. 1c, an alternative, or further, embodiment of the contrast agent mixer 100 is illustrated. In this embodiment, the holding arrangement 109 is movable along the longitudinal axis L of the mixer shaft 120. The movement of the holding arrangement 109 may be controlled by the second motor arrangement 140 similarly to the movement of the mixer blade 110 along the longitudinal axis L, e.g., by means of rotating of motor shaft 14.

In FIG. 1d, a side view of the mixer 100 according to some embodiments wherein the connection between the motor shaft 145 and the holding arrangement 109 is visible. The holding arrangement 109 is in this embodiment movable along the column 105 of the mixer such that the distance D between the holding arrangement 109 and the mixer blade 110 is changed.

It should be mentioned that in some embodiments (not shown), the second motor arrangement 140 may be configured to control a vertical positon of the both the holding arrangement 109 and the mixer blade 110. This may be provided by the second motor arrangement 140 being configured to move one of the mixer blade 110 and the holding arrangement in a first vertical direction, and the other of the motor arrangement 140 and the mixer blade 110 in a second vertical direction, the second vertical direction being opposite the first vertical direction. In some embodiments, one or more clutch arrangements are provided between the second motor arrangement 140 and one or both of the mixer blade 110 and the holding arrangement 109. In such embodiments, the second motor arrangement 140 may be configured to selectively control the vertical positon of the mixer blade 110 and/or the holding arrangement 109.

In FIG. 1e, another embodiment of the contrast agent mixer 100 is shown in a side view corresponding to that of FIG. 1b. In this embodiment, the contrast agent mixer is similar to the contrast agent mixer 100 of the previous embodiment in every aspect except that it does not comprise a movable holding arrangement 109. It may comprise a fixed holding arrangement (not shown). This means that the second motor arrangement 140 controls a vertical positon of the mixer blade 110 along the longitudinal axis L. This implies that, when the vertical position of the mixer blade 110 is controlled, the first motor arrangement 130 is moved together with the mixer blade 110.

It should be mentioned that by changing the distance D between the holding arrangement 109 and the mixer blade 110 by moving the holding arrangement 109 along the longitudinal axis of the mixer shaft 120 may be beneficial as it reduces vibrations and noise. This is due to e.g., that the rotatable mixer blade 110 may be more securely fastened in this embodiment. However, changing the distance D between the holding arrangement 109 and the mixer blade 110 by moving the mixer blade 110 along the longitudinal axis of the mixer shaft 120 may be beneficial as the second motor arrangement 140 may be reduced in size and weight and thereby reducing cost. This is due to e.g., that the holding arrangement 109 together with the mixing container 200 with liquid 3 and powder 5 is, in most embodiments, heavier than the corresponding movable parts associated with the mixer blade.

The second motor arrangement 140 may be a stepper motor 140. The first motor arrangement 130 may be a brushless DC motor 130.

It should be emphasized that although the embodiment in FIGS. 1a and 1b is described as the mixer blade 110 being movable along the longitudinal axis L of the mixer shaft 120, and the embodiment in FIGS. 1c and 1d is described as the holding arrangement 109 being movable along the longitudinal axis L of the mixer shaft 120, these embodiments are not mutually exclusive. The skilled person will understand that embodiments wherein both the mixer blade 110 and the holding arrangement 109 are movable along the longitudinal axis L and that the second motor arrangement 140 may be configured with e.g., gearing and/or clutches to facilitate this mutual movement. In summary, either one of or both of the mixer blade 110 and the holding arrangement 109 are movable along the longitudinal axis L such that the distance D between the holding arrangement 109 and the mixer blade 110 is changed. It should be mentioned that in embodiments wherein the mixer 100 does not comprise a specific holding arrangement 109, movable or stationary, the distance D between the holding arrangement 109 and the mixer blade 110 is to be interpreted as a distance between the mixer blade 110 and a surface for supporting the mixing container 200. Such a surface may be e.g., the base 103 or a table on which the mixer 100 is placed.

The mixer shaft 120 may in some embodiment be connected to the first motor arrangement 130 by means of a clutch 150. The clutch 150 is beneficial as it may be configured to allow simple and quick connection and disconnection of the mixer blade 110 to the mixer 100. If, for instance, the mixer blade 110 is reusable, it may be easily removed and cleaned. Additionally, if the mixer blade 110 is disposable, it may be easily removed and replaced. It should be mentioned that the mixer blade 110 may, depending on embodiment, be interpreted as comprising also the mixer shaft 120. In one embodiment, the mixer blade 110 is a stainless steel mixer blade 110 which is beneficial as it is easy to clean and durable. In another embodiment, the mixer blade 110 is a plastic mixer blade 110 which is beneficial as it reduces the need of cleaning.

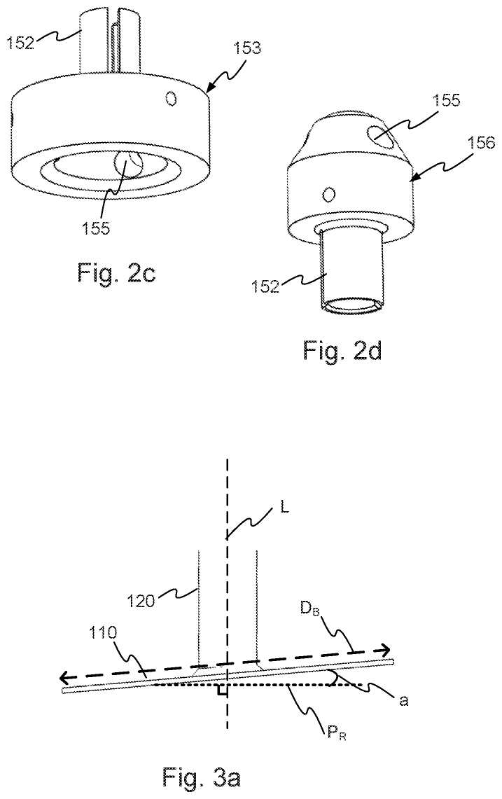

The clutch 150 may be formed in any suitable way that allows release of the mixer blade 110 from the first motor arrangement 130. With reference to FIGS. 2a to 2d, one preferred embodiment of the clutch 150 will be explained in further detail. In this embodiment, the clutch 150 comprises an upper member 153 and a lower member 156. The upper member 153 is operatively connected to the first motor arrangement 130 and the lower member 156 operatively connected to the mixer blade 110. The operative connection of the lower member 156 to the mixer blade 110 may be via the mixer shaft 120. The operative connection of the upper member 153 to the first motor 130 may comprise a drive shaft 135 of the first motor arrangement 130. The upper member 153 and the lower member 156 may, as best illustrated in the cross sectional view of FIG. 2b, be connected to respective shaft 135, 120 by any suitable fastening means 152 or formed integral with the shafts 135, 120. The upper member 153 and the lower member 156 are detachably attached to each other by one or more attachment means 155, see FIGS. 2c and 2d illustrating the upper member 153 and the lower member 156 separated. The attachment means 155 may be any suitable attachment means 155 such as a bayonet mount, a click mount, nut and bolt configuration etc. One of the upper member 153 or the lower member 156 may be formed with guiding protrusions (not shown) and the other of the upper member 153 and the lower member 156 with mating notches (not shown).

However, in order to keep the surfaces of the upper member 153 and the lower member 156 smooth and avoid difficult cleaning and buildup of dirt, the attachment means 155 are in a preferred embodiment formed as magnets 155. The attachment means 155 may be one single magnet 155 provided in either of the upper member 153 or the lower member 156 provided that the other of the upper member 153 or the lower member 156 is magnetic. As the upper member 153 will transfer rotation about the longitudinal axis L of the mixer shaft 120, the clutch 150 will be subjected to torque; and the attachment means 155 has to be sufficiently strong to withstand this. The torque, and also Euler forces, subjected to the clutch 150 will depend on an acceleration provided by the first motor arrangement 130. A comparably weaker attachment means 155 may be compensated by configuring the controller 160 to reduce the torque exerted by the first motor 130. The inventors behind this disclosure have realized that a sufficiently strong clutch 150 is provided by arranging a plurality of magnets 155 at each of the upper member 153 and the lower member 156. Preferably the magnets 155 are embedded in cavities of the upper member 153 and the lower member 156 and arranged such that, when the clutch 150 is assembled, a south pole of magnets 155 in the upper member 153 face a north pole of magnets 155 the lower member 156 or vice versa. In a preferred embodiment, the upper member 153 and the lower member 156 is provided with three magnets each.

The magnets 155 and their associated cavities are preferably covered with a suitable coating to reduce the presence of hard to clean nooks and crannies.

As seen in the cross sectional view of the clutch 150 as presented in FIG. 2b and in the isolated views of the upper member 153 in FIG. 2c and the lower member 156 in FIG. 2d, the lower member 156 may be formed with a convex shape and the upper member 153 with a mating concave shape. Although not illustrated, the opposite setup is also possible wherein the upper member 153 is formed with a convex shape and the lower member 156 with a mating concave shape although the convex shape is easier to clean and preferred for the lower member 156.

It should be noted that albeit shown together, the attachment means 155 provided as magnets 155 and the shape of the upper member 153 and the lower member 156 are not directly linked and e.g., any suitable attachment means 155 may be combined with any suitable shape of the upper member 153 and the lower member 156.

Additionally, or alternatively, as previously mentioned, the mixer blade 110 may be removable from the mixer shaft 120. In such embodiments, the mixer blade 110 may be attached to the mixer shaft 120 by means of e.g., a twist lock, a snap-in or other suitable attachment means. Such arrangements are beneficial as the mixer blade 110 and the mixer shaft 120 may be provided from different materials. In one embodiment, the mixer blade 110 is a plastic mixer blade 110 and the mixer shaft 120 is a metal mixer shaft. The mixer blade 110 may be a disposable mixer blade 110 and the mixer shaft 120 may be a reusable mixer shaft 120.

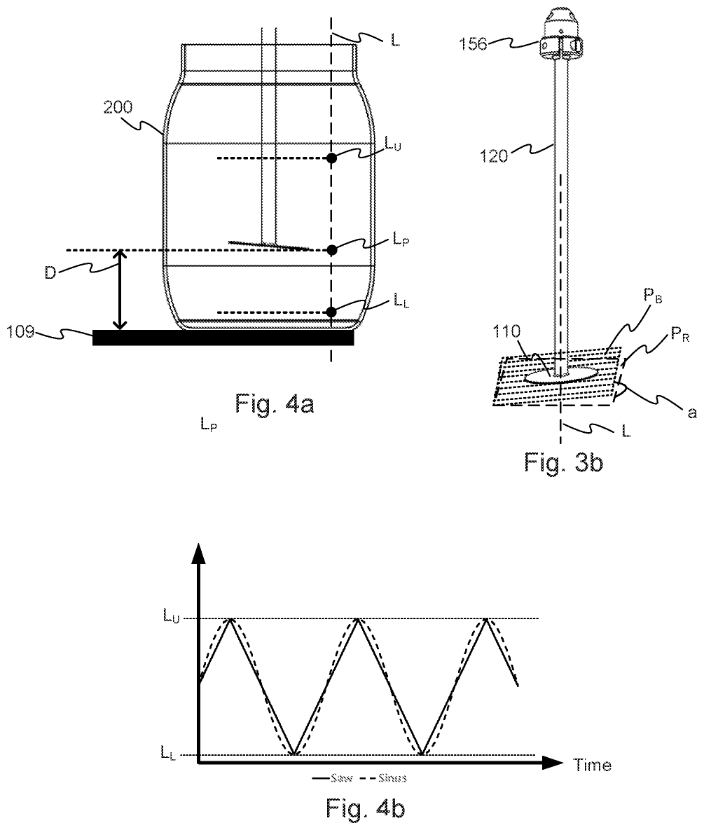

With reference to FIGS. 3a and 3b, in order to ensure a consistent and efficient foaming of the negative contrast agent foam 7, the inventors behind this disclosure has, through inventive thinking, concluded that the mixer blade 110 is preferably arranged on the mixer shaft 120 such that a blade angle a is formed between a plane P_{B} of the mixer blade 110 and a reference plane P_{R}. Wherein the reference plane P_{R} is perpendicular to the longitudinal axis A of the mixer shaft 120. A large blade angle a will incorporate more air compared to a small blade angle a and thus provide a larger foam 7 volume assuming all other conditions are the same. Substantive research and experimenting has concluded that a blade angle a in the range of 0.5 to 5° provides an acceptable per-oral negative contrast agent foam 7 for abdominal CT. If the blade angle a is within the range of 2 to 4°, a better contrast agent foam 7 is provided and a blade angle at substantially 3° has been shown to be most preferable.

The mixer blade 110 may be formed in various shapes, but experimental tests and research has concluded that a homogenous mixer blade 110 provides a suitable negative contrast agent foam 7. Adding holes or cavities to the mixer blade 110 reduces the effect of the mixing blade 110 as the increased turbulence caused by the holes provides a foam 7 that is less homogeneous and with many visible bubbles compared to a homogenous mixer blade 110. Further to this, the mixer blade 110 may be formed in a substantially circular circumferential shape, preferably with the mixer shaft 120 centered on the mixer blade 110 as this provides a balanced load and reduces the risk of vibrations when the mixer blade 110 is rotated. A lower surface of the mixer blade 110 may comprise a bulge, or a stud. The lower surface may be saucer shaped. This is beneficial as it facilitates more turbulence during operation of the mixer 100, which is advantageous for mixing and foam formation.

With reference to FIGS. 4a and 4b, the movement of the mixer blade 110 and/or the holding arrangement 109 along the longitudinal axis L will be explained in further detail. The mixer blade 110 and/or the holding arrangement 109 is preferably moved repeatedly up and down such that a vertical distance L_{P} between the mixer blade 110 and the holding arrangement 109 is shifted between an upper distance L_{U} and a lower distance L_{L} along the longitudinal axis L. This may be provided by the controller 160 controlling the second motor arrangement 140 to move the mixer blade 110 and/or the holding arrangement 109 up and down along the longitudinal axis L of the mixer shaft 120. At the lower distance L_{L}, the mixer blade 110 is preferably located close to a bottom of the mixing container 200 but may for various reasons be distanced from the bottom of the mixing container 200. In some embodiments, at the upper distance L_{U}, the mixer blade 110 is fixed at a height of approximately half the height of the mixing container 200. In other embodiments, the upper distance L_{U} between the mixer blade 110 and the holding arrangement 109 is determined based on a wanted height of the negative contrast agent foam 7 in the mixing container 200. In one embodiment, the upper distance L_{U} of between the mixer blade 110 and the holding arrangement 109 is below 75% of a wanted height of the negative contrast agent foam 7, preferably below 65% of the wanted height of the negative contrast agent foam 7, and most preferably below 55% of the wanted height of the negative contrast agent foam 7 in the mixer container 200. In some embodiments, the upper distance L_{U} is adapted based on a current height of the negative contrast agent foam 7 in the mixing container 200. The upper distance may be adapted to be below 75%, preferably below 65%, and most preferably below 55% of the current height of the negative contrast agent foam 7 in the mixer container 200.

As seen in FIG. 4b, the controlling 320 of the vertical distance L_{P} between the mixer blade 110 and the holding arrangement 109 may be done with a substantially constant movement such that the vertical distance L_{P} between the mixer blade 110 and the holding arrangement 109 plotted over time will describe a sawtooth curve indicted by the solid line of FIG. 4b. Alternatively, the controlling 320 of the vertical distance L_{P} between the mixer blade 110 and the holding arrangement 109 may be done with a substantially sinusoidal movement such that the vertical distance L_{P} between the mixer blade 110 and the holding arrangement 109 plotted over time will describe a sinusoidal curve, indicated by the dotted line of FIG. 4b.

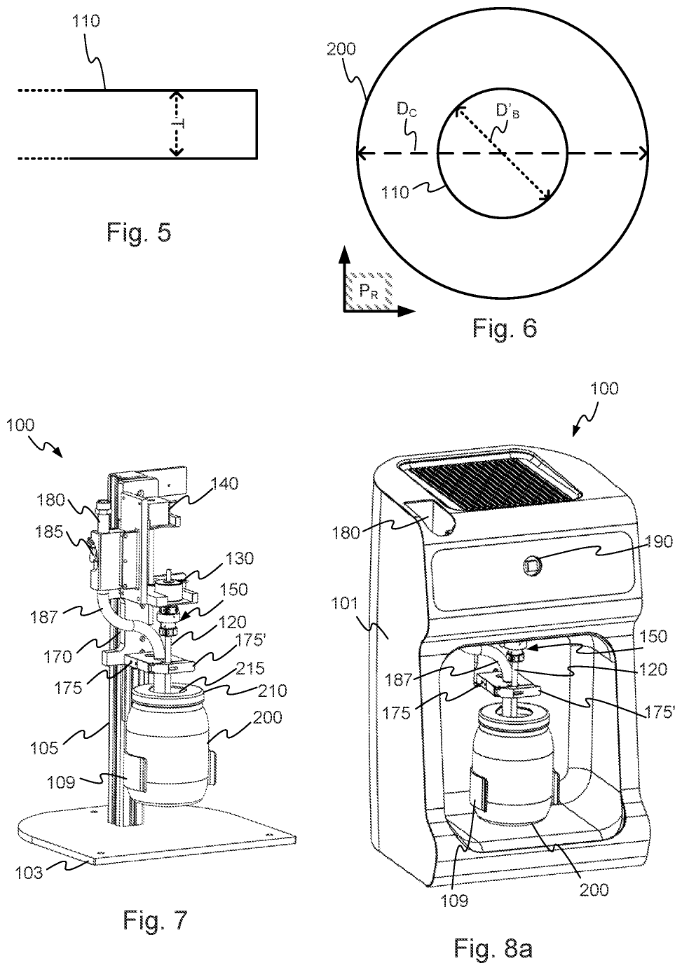

Through further research and experimental efforts, the inventors behind this disclosure have concluded that there are further design features of the mixer blade 110 that may be utilized to further improve the quality of the negative contrast agent foam 7. In FIG. 5, a vertical thickness T of the mixer blade 110 is indicated and the mixer blade thickness T has been shown to affect the size of the bubbles of the negative contrast agent foam 7. Reducing the thickness T of the mixer blade 110 will reduce the size of the bubbles. Reducing the thickness T too much may make the mixer blade 110 too flexible and it may bend or otherwise deformed during handling. The mixer blade 110 is in one embodiment configured with a thickness T of 0.4 to 1.8 mm, and in a further embodiment with a thickness of 0.5 to 1.1 mm and in an even further embodiment, a thickness T of from (and including) 0.7 to (and including) 1.0 mm. It should be noted that the size of the bubbles of the foam 7 will depend on other factors such as the rotational speed of the mixer blade 110.

With reference to FIG. 6, one further feature of the present disclosure will be explained. In order to incorporate a controllable volume of air when mixing the dry powder 5 and the liquid 3, a size of the mixer blade 110 in relation to the mixing container 200 is preferably controlled. FIG. 5 is a top view of the mixing container 200 with the mixer blade 110 substantially centered in the mixing container 200. The mixer blade 110 exhibits a projected diameter D′_{B} in the reference plane P_{R}. That is, assuming a circular mixer blade 110 having a diameter D_{B} (see FIG. 3a), this would result in a projected diameter D′_{B} of the mixer blade 110 in the reference plane P_{R} equal to the diameter D_{B} of the mixer blade 110 multiplied by the cosine value of the blade angle a, D′_{B}=D_{B}·cos a). The mixing container 200 correspondingly exhibits a diameter D_{C} in the reference plane P_{R}. Research and experimental efforts by the inventors of this disclosure have resulted in the teaching that a ratio of the diameter D_{C} of the mixing container 200 and the projected diameter D′_{B} of the mixer blade 110 in the reference plane P_{R}, D_{C}/D′_{B}, of between 2 and 3 allows for a good incorporation of air in the foam 7. Preferably, the diameter D_{C} of the mixing container 200 is between 2.4 and 2.7 times larger than the projected diameter D′_{B} of the mixer blade 110 in the reference plane P_{R}, most preferable about 2.5 times.

Generally, a diameter ratio between the mixer blade 120 and the mixing container 200 may be in the range of 0.3 to 0.7, preferably around 0.4. As an example, the diameter D_{B} of the mixer blade 120 may be in the range of 35 mm to 60 mm, preferably 40 mm to 55 mm, more preferably 48 mm, and the diameter D_{C} of the mixing container 200 may be 80-200 mm, preferably between 100-130 mm. A diameter of a bottom of the mixing container bottom may however be smaller, for instance 40-70 mm.

In one embodiment of the negative contrast agent mixer 100, presented in a perspective view in FIG. 7, it further comprises a liquid container 180 arranged to dispense the liquid 3 into the mixing container 200. The liquid container 180 may be of any suitable shape, size or form and is not limited to the tubular shape as illustrated in FIG. 7. The liquid container 180 is preferably operatively connected to a tube member 187 arranged to guide the liquid 3 from the liquid container 180 to the mixing container 200. In one further embodiment, a valve 185 is arranged to control the flow of liquid 3 from the liquid container 180 into the mixing container 200. The valve 185 may be arranged between the liquid container 180 and the tube member 187. The valve 185 is controllable between an open and a closed position and in one embodiment, the controlling of the position of the valve 185 is provided by the controller 160. The valve 185 may be controllable in one or more discrete steps or continuously and step-less between the open position and the closed position. The liquid container 180 may optionally be provided with one or more sensors (not shown) configured to detect a presence of liquid 3 in the liquid container 180.

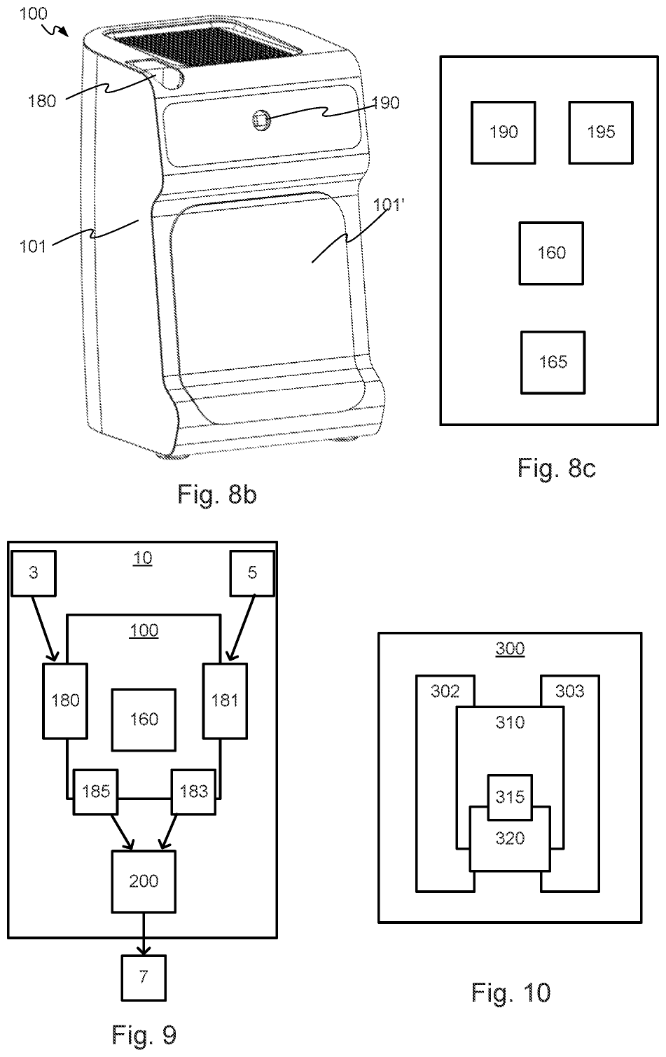

In alternative, or additional, embodiments of the mixer 100, it may further comprise a powder container 181 (see FIGS. 8a-b) arranged to dispense the contrast powder 5 into the mixing container 200. In one embodiment, the powder container 181 is pivotably connected to the mixer 100 and controllable between a tilted position, at which the contrast powder 5 is dispensed into the mixing container 200, and an upright position, at which the contrast powder 5 is stayed in the powder container 181. The control of the powder container 181 is preferably provided by the controller 160. The powder container 181 may also be operatively connected to a tube member (not shown) for guiding the powder into the mixing container. The release of the contrast powder 5 into the tube member may be controlled by e.g., a hinged trap-door configuration. The pivoted connection and the hinged trap-door configuration are referred to as a powder dispenser 183, see FIG. 9. The powder dispenser 183 is consequently arranged between the mixing container and the powder container 181 in a path of the contrast powder 5.

FIG. 7 further illustrates a clip member 109 of the holding arrangement 109 that in this embodiment is attached to the column 105. It should be emphasized that, as previously explained, the holding arrangement 109 may alternatively be connected to the second motor arrangement 140 such that it is movable along the longitudinal axis L of the mixer shaft 140. However, in embodiments in which only the mixer blade 110 is movable along the longitudinal axis L of the mixer shaft L, the holding arrangement may e.g., be attached to the column 105 as illustrated in FIG. 7, or form part of the base 103. The clip member 109 is usable to hold the mixing container 200 in position during operation of the mixer 100.

In order to ensure that the mixer shaft 120 is in position and correctly balanced, the carrier 170 may be provided with a guide member 175 arranged distanced from the first motor arrangement 130 and the clutch 150. The guide member 175 is provided to guide the mixer shaft 120 and to ensure its centration in the mixing container 200. The guide member 175 may be provided with a locking member 175′ arranged to secure the mixer shaft 120 in e.g., a notch in the guide member 170 such that the mixer shaft 120 may rotate freely about the longitudinal axis L of the mixer shaft 120 but it is not permitted to form an angle to the longitudinal axis L. The locking member 175′, the guide member 175 and/or a notch of the locking member 175′ and/or the guide member 175 may be provided with bearings or other suitable friction reducing means allowing the mixer shaft 120 to rotate freely even though it is guided by the guide member 175 and optionally the locking member 175′.

The mixing container 200 as illustrated in FIG. 7 further comprises a removable lid 210. This lid 210 is provided to reduce spill and splatter of the liquid 3 and/or the foam 7. The lid 210 is provided with an opening 215 configured to allow the mixer blade 110 to enter the mixing container 200.

It should be mentioned that the mixing container 200 may be any vessel suitable for holding the liquid 3, the powder 5 and allowing them to be mixed into a foam. The mixing container 200 may be a glass, metal or plastic container and in a preferred embodiment, the mixing container is a paper material mixing container 200.

In further embodiments, the guide member 175 may further be configured to form a guide for the tube member 178 for the liquid container 180 and/or the powder container 181.

In FIGS. 8a and 8b, perspective views of the mixer 100 is presented illustrating an embodiment where the mixer 100 comprises an outer housing 101. The outer housing 101 may be made from any suitable material and in one embodiment the outer housing 101 is a plastic casing allowing the mixer 100 to, in at least some countries, be powered by main power without a need for protective ground. The outer housing 101 makes the mixer 100 esthetically pleasing, dampens sounds from the mixer 100, reduced the risk of splashes, provides protection for the mixer 100 etc. The outer housing 101 is preferably provided with an openable door 101′, illustrated in FIG. 8b, that may be opened to provide access to the mixing container 200 and the mixer blade 120. The liquid container 180 is preferably accessible from outside the outer housing 101. The door 101′ may of a different type of material than the other parts of the outer housing 101. In one embodiment, the door 101′ is a transparent plastic door 101′ allowing a user of the mixer 100 to see the forming of the foam 7.

The mixer 100 may be provided with a user interface 190. The user interface 190 may be controlled by the controller 160 and may be used to communicate operational data to a user of the mixer 100 and also to receive operational data from the user. In one embodiment, the user interface 190 is an illuminated button 190. When the button 190 is pressed, the mixer 100 is started and a color of the illumination indicate the status of the mixer 100. Different illumination colors and patterns may be utilized to indicate different states and/or different requested actions from the user. Such states and actions may be, but are not limited to, an off state where the mixer 100 is unpowered, an idle state where the mixer 100 is ready to use, a state wherein the door 101′ is open, a state wherein the mixing container 200 is missing, a state wherein the liquid container 180 is empty, a state wherein the powder container 181 is empty, a mixing state wherein mixing is in process, a mixing completed state etc.

As illustrated in FIG. 8c, a partial block diagram of the mixer 100 according to some embodiments, the mixer 100 may be provided with one or more external interfaces 195. These external interfaces 195 may be one or more of any suitable wired or wireless interfaces e.g., serial interfaces (RS232, USB etc.), parallel interface (IEEE 1284 etc.), WiFi, cellular interface (GSM, UMTS, LTE, NR etc.), Bluetooth (BLE), low power WAN (LoRa, Sigfox etc.), etc. The external interface 195 may be configured to allow the mixer to be connected to other devices directly or via e.g., a cloud service forming part of an Internet of Things, IoT network. The mixer 100 may be configured to, preferably by means of the controller 160, to communicate with a server in order to share a current operational status, receive control commands and/or receive software or configurational updates. The current operational status may comprise one or more of a number of mixing cycles performed, any error messages, current amount of liquid in the liquid container 180, current amount of powder in the liquid container 180, any need for maintenance etc. The data provided by the mixer 100 may be used to schedule maintenance of the mixer 100, place orders for powder and/or liquid etc. The control commands received through the external interface 195 may be service commands, start commands, stop commands etc. The control commands may be provided from a cloud service or directly to the mixer via e.g., Bluetooth. The external interface allows for remote and/or touchless control of the mixer by e.g., a mobile device.

The mixer 100 may further be provided with one or more sensors 165. Sensors 165 may be provided to detect an amount of liquid 3 added to the mixing container 200, an amount of contrast powder 5 added to the mixing container 200, a status of the foaming of the negative contrast agent foam 7, a height of the foam 7 in the container 200, a weight of the container 200, a level of liquid 3 in the liquid container 180, a level of contrast powder 5 in the powder container 181, a rotational speed of the mixer blade 110, a presence of the mixer blade 110, a closure of the locking member 175′, a presence of the mixing container 200, the vertical distance L_{P} between the mixer blade 110 and the holding arrangement 109 etc. The sensors 165 may be any type of suitable sensor 165 such as, but not limited to, switches, optical sensors, pressure sensors, ultrasonic sensors, accelerometers, current sensors, voltage detectors etc. The controller 160 is preferably operatively connected to the sensors 165 and configured to control the operation of the mixer 100 based on data provided by the sensors 165.

In one embodiment, the controller 160 is configured to control, based on data from one or more sensors 165, an amount of liquid 3 added to the mixing container 200. In a further, or alternative embodiment, the controller 160 is configured to control, based on data from one or more sensors 165, an amount of powder 5 added to the mixing container 200.

With reference to FIG. 9, a mixer system 10 is shown. The mixer system comprises the negative oral contrast agent mixer 100 as presented herein, the mixing container 200 as previously introduced, the contrast powder 5 and the liquid 3. As presented, the mixing container 200 is arrangeable to receive the mixer blade 110 of the mixer 100. The liquid 3 may be provided in the liquid container 180 and dispersed in the mixing container 200 under control of the controller 160. The contrast powder 5 may be provided in the powder container 181 and dispersed in the mixing container 200 under control of the controller 160. The mixer system 10 is configured to provide the per-oral negative contrast agent foam 7 for abdominal CT as described herein.

With reference to FIG. 10 a method 300 for providing a per-oral negative contrast agent foam 7 for abdominal CT will be presented. The method 300 may be performed by any suitable means configured to control a vertical distance L_{P} between a mixer blade and a holding arrangement and the rotational speed of the mixer blade, but is preferably performed by the controller 160 of the negative oral contrast agent mixer 100 as disclosed herein. The mixer blade 110 is configurable to mix the contrast powder 5 with the liquid in the mixing container 200. The method 300 comprises controlling 310 the mixer blade 110 to rotate at a wanted rotational speed. This may be provided by the controller 160 controlling the first motor arrangement 130 to rotate at the wanted rotational speed. It may further comprise accelerating, for a predetermined or configurable acceleration time period, the rotational speed mixer blade 110 until it reaches the wanted rotational speed. This is beneficial as it reduces the wear of the first motor arrangement 130 and also the risk of splashes of the liquid due to sudden changes in rotational speed of the mixer blade 110. In one embodiment, the wanted rotational speed is in the range of 6500 to 10000 rpm, preferably in the range of 8000 to 8500 rpm. It should be mentioned that the wanted rotational speed may very well be different at different stages of the method 300. In one embodiment, the rotational speed of the mixer blade 110 is lower at a start of the method 300 than at the end of the method 300.

As the dispersion will have a different viscosity compared to the negative contrast agent foam 7, it is beneficial to control 310 a current of the first motor arrangement 130 when controlling the rotational speed of the mixer blade 110. This ensures a constant rotational speed of the first motor arrangement 130 regardless of the load presented to the mixer blade 110.

The method 300 further comprises controlling 320 the vertical distance L_{P} between the mixer blade 110 and the holding arrangement to repeatedly change between the upper distance L_{U} and the lower distance L_{L} along the longitudinal axis L of the mixer shaft 120. This may be provided by the controller 160 controlling the second motor arrangement 140 to move the mixer blade 110 and/or the holding arrangement 109 up and down along the longitudinal axis L of the mixer shaft 120. The controlling 320 of the vertical distance L_{P} between the mixer blade 110 and the holding arrangement may, as described in reference to FIG. 4b, be done with a substantially constant movement such that the vertical distance L_{P} between the mixer blade 110 and the holding arrangement 109 plotted over time will describe a sawtooth curve. Alternatively, the controlling 320 of the vertical distance L_{P} between the mixer blade 110 and the holding arrangement 109 may be done with a substantially sinusoidal movement such that the vertical distance L_{P} between the mixer blade 110 and the holding arrangement 109 plotted over time will describe a sinusoidal curve.

It should be mentioned that controlling 320 the vertical distance L_{P} between the mixer blade 110 and the holding arrangement 109 to repeatedly move between the upper distance L_{U} and the distance L_{L} may be performed a predefined or configurable number of times. In one embodiment of the method 300, the vertical distance L_{P} between the mixer blade 110 and the holding arrangement 109 is cycled between the upper distance L_{U} and the lower distance L_{L} at least 8 times, and in a preferred embodiment, the vertical distance L_{P} between the mixer blade 110 and the holding arrangement 109 is repeated at least 12 times. Alternatively, or additionally, the vertical distance L_{P} between the mixer blade 110 and the holding arrangement 109 is cycled between the upper distance L_{U} and the lower distance L_{L} less than 25 times, and in a preferred embodiment, the movement of the vertical distance L_{P} between the mixer blade 110 and the holding arrangement 109 is cycled less than 17 times. The speed of the vertical movement of the mixer blade 110 and/or the holding arrangement 109 is preferably such that the desired number of cycles is executed in less than 4 minutes, preferably in between 2 and 3 minutes.

In some embodiments, the wanted rotational speed of the mixer blade 110 is different depending on the vertical distance L_{P} between the mixer blade 110 and the holding arrangement 109. In a preferred embodiment, the wanted rotational speed of the mixer blade 110 is lower at the lower distance L_{L} than at the upper distance L_{U} between the mixer blade 110 and the holding arrangement 109.

In embodiments of the mixer 100 wherein it comprises the liquid container 180 and the valve 185, the method 300 may further comprise controlling 302 the valve 185 to dispense liquid 3 into the mixing container 200. The controlling 302 of the valve 185 may be done subsequent to initiating the control 310 of the mixer blade 110 to rotate at the wanted rotational speed or before initiating the control 310 of the mixer blade 110 to rotate at the wanted rotational speed.

Similarly, in embodiments of the mixer 100 wherein it comprises the powder container 181 and the powder dispenser 183, the method 300 may further comprise controlling 303 the powder dispenser 183 to dispense the contrast powder 5 into the mixing container 200. The controlling 303 of the powder dispenser 183 may be done subsequent to initiating the control 310 of the mixer blade 110 to rotate at the wanted rotational speed or before initiating the control 310 of the mixer blade 110 to rotate at the wanted rotational speed.

In order to reduce the risk of buildup of large bubbles of air in the foam 7, it may be beneficial to pause the rotation of the mixer blade 110 for a period to allow any oversized bubbles to raise to a surface of the foam 7 and collapse. This may be provided by stopping 315 the rotation of the mixer blade 110 for a rest period.

The method 300 may, as the skilled person will understand after digesting the teachings of this disclosure, be modified to comprise reading data from any of the sensors mentioned in this disclosure. The method 300 may comprise ensuring that liquid 3, contrast powder 5, the mixing container 200 etc. is present before initiating the mixing. The method 300 may be executed until a sensor indicate that sufficient foaming is accomplished.

Modifications and other variants of the described embodiments will come to mind to one skilled in the art having benefit of the teachings presented in the foregoing description and associated drawings. Therefore, it is to be understood that the embodiments are not limited to the specific example embodiments described in this disclosure and that modifications and other variants are intended to be included within the scope of this disclosure. For example, while embodiments of the invention have been described with reference to a negative oral contrast agent mixer with related methods and systems, persons skilled in the art will appreciate that the embodiments of the invention can equivalently be applied to mixing of other agents where a homogenous and controlled foaming is desired. Furthermore, although specific terms may be employed herein, they are used in a generic and descriptive sense only and not for purposes of limitation. Therefore, a person skilled in the art would recognize numerous variations to the described embodiments that would still fall within the scope of the appended claims. Furthermore, although individual features may be included in different claims (or embodiments), these may possibly advantageously be combined, and the inclusion of different claims (or embodiments) does not imply that a combination of features is not feasible and/or advantageous. In addition, singular references do not exclude a plurality. Finally, reference signs in the claims are provided merely as a clarifying example and should not be construed as limiting the scope of the claims in any way.

1. A contrast agent mixer for providing a foam type contrast agent, the contrast agent mixer comprising a holding arrangement for supporting a mixing container, a substantially homogeneous circular mixer blade for mixing a contrast powder with a liquid in the mixing container, and a controller configured to control a rotational speed of the mixer blade to a wanted rotational speed and a vertical distance between the holding arrangement and the mixer blade.

2. The contrast agent mixer of claim 1, wherein the holding arrangement is movable by a second motor arrangement of the contrast agent mixer to control the vertical distance between the holding arrangement and the mixer blade, the second motor arrangement is controlled by the controller.

3. The contrast agent mixer of claim 1, wherein the mixer blade is arranged on a mixer shaft rotatable about a longitudinal axis of the mixer shaft by a first motor arrangement controlled by the controller.

4. The contrast agent mixer of claim 1, wherein the mixer blade is movable by the second motor arrangement of the contrast agent mixer to control the vertical distance between the holding arrangement and the mixer blade, the second motor arrangement is controlled by the controller.

5. The contrast agent mixer of claim 3, wherein the mixer blade is operatively connected to the first motor arrangement by means of a clutch,
wherein the clutch is a magnetic clutch comprising an upper member operatively connected to the first motor arrangement and a lower member operatively connected to the mixer blade, wherein the upper member is connected to the lower member by means of one or more magnets.

6. (canceled)

7. The contrast agent mixer of claim 5, wherein the lower member or the upper member of the clutch is concavely formed, and the other of the lower member or the upper member of the clutch is matingly convexly formed.

8. The contrast agent mixer of claim 3, wherein the mixer blade is arranged on the mixer shaft such that a blade angle is formed between a plane of the mixer blade and a reference plane perpendicular to a longitudinal axis of the mixer shaft, wherein the blade angle is in the range of 0.5 to 5°, preferably in the range of 2 to 4°.

9. The contrast agent mixer of any of claim 1, further comprising a liquid container arranged to dispense the liquid into the mixing container and a valve arranged in a fluid pathway between the liquid container and the mixing container and controllable between an open position and a closed position by the controller.

10. (canceled)

11. The contrast agent mixer of claim 1, further comprising a powder container arranged to dispense the contrast powder into the mixing container.

12. A method for providing a per-oral negative contrast agent foam for abdominal computer tomography, CT, performed by the controller of the contrast agent mixer of claim 1, wherein the mixer blade is configurable to mix a contrast powder with a liquid in a mixing container, the method comprising:
controlling the mixer blade to rotate at a wanted rotational speed, and
controlling a vertical distance between the mixer blade and the holding arrangement to repeatedly change between an upper distance and a lower distance.

13. The method of claim 12, wherein the contrast agent mixer comprises a liquid container arranged to dispense liquid into the mixing container by means of a valve, the method further comprising:
controlling the valve to dispense liquid into the mixing container.

14. The method of claim 13, wherein controlling the mixer blade to rotate at the wanted rotational speed is initiated before controlling the valve to dispense liquid into the mixing container.

15. The method of claim 13, wherein controlling the contrast agent mixer blade to rotate at the wanted rotational speed is initiated after controlling the valve to dispense the liquid into the mixing container.

16. The method of claim 12, wherein controlling the vertical distance between the mixer blade and the holding arrangement to repeatedly change between the upper distance and the lower distance is performed at least 8 times, preferably at least 12 times.

17. The method of claim 12, wherein controlling the mixer blade to rotate at the wanted rotational speed further comprises, during controlling the vertical distance between the mixer blade and the holding arrangement:
stopping the rotation of the mixer blade for a rest period.

18. A mixer system comprising the contrast agent mixer of claim 1, a mixing container, a contrast powder and a liquid, wherein the mixing container is arrangeable to receive the mixer blade of the mixer.

19. The mixer system of claim 18, wherein a diameter of the mixer blade is less than half a diameter of the mixing container and larger than one third of the diameter of the mixing container.

20. The mixer system of claim 18, wherein the vertical distance between the mixer blade and the holding arrangement is controllable between an upper distance and a lower distance, the mixer blade being adjacent to a bottom of the mixing container at the lower distance.

21. The mixer system of claim 20, wherein the upper distance between the mixer blade and the holding arrangement is, during operation of the mixer, below 75% of a wanted height of the negative contrast agent foam in the mixer container, preferably below 65% of the wanted height of the negative contrast agent foam in the mixer container, and most preferably below 55% of the wanted height of the negative contrast agent foam in the mixer container.

22. The mixer system of claim 18, wherein the contrast powder is an egg albumen powder and the liquid is water.

23. (canceled)
